(19) United States
(12) Patent Application Publication
(10) Pub. No.: US 2010/0068786 A1
(43) Pub. Date: Mar. 18, 2010

(54) METHODS AND COMPOSITIONS FOR REVERSING P-GLYCOPROTEIN MEDICATED DRUG RESISTANCE

(76) Inventors: Jean A. Chmielewski, Lafayette, IN (US); Christine A. Hrycyna, Lafayette, IN (US); Marcos M. Pires, West Lafayette, IN (US)

Correspondence Address: BOSE MCKINNEY & EVANS LLP, 111 MONUMENT CIRCLE, SUITE 2700, INDIANAPOLIS, IN, 46204, US

(21) Appl. No.: 12/561,014

(22) Filed: Sep. 16, 2009

Related U.S. Application Data
(60) Provisional application No. 61/097,308, filed on Sep. 16, 2008.

(51) Int. Cl. C12N 9/16; C07H 19/06; C07D 221/28; C07D 233/02; C07D 215/42

(52) U.S. Cl. 435/196; 435/325; 536/28.4; 536/28.54; 546/74; 548/321.1; 564/360

(57) ABSTRACT

A method for inhibiting therapeutic drug resistance within a cell over-expressing a membrane protein is provided. The method comprises synthesizing a dimeric prodrug inhibitor of a monomeric therapeutic agent; administering the dimeric prodrug inhibitor to the membrane protein together with the monomeric therapeutic agent; and occupying at least one substrate binding site of the membrane protein with the synthesized dimeric prodrug to allow the monomeric therapeutic agent to accumulate within the cell. The dimeric prodrug inhibitor contains a crosslinking agent that is adapted to breakdown under reducing conditions within the cytosol of the cell to cause the dimeric prodrug to revert back to a form equivalent to the monomeric therapeutic agent.

### RELATED APPLICATIONS

This application claims the benefit of U.S. Provisional Patent Application Ser. No. 61/097,308, filed Sep. 16, 2008, the complete disclosure of which is expressly incorporated herein by this reference

This invention was made in part with government support under grant reference number 102678 awarded by the National Institutes of Health (“NIH”) and sponsored by the National Eye Institute under Sponsor Award No.: 1R21EY018481-01. The Government therefore has certain rights in this invention.

### TECHNICAL FIELD

The teachings of the present invention generally relate to methods and compositions for blocking drug resistant proteins, and more particularly to using prodrug dimers of known drugs to avoid drug resistance.

### BACKGROUND OF THE INVENTION

Despite positive developments in drug therapies for brain disorders such as depression, epilepsy, schizophrenia/psychosis, Alzheimer's disease and drug and alcohol addiction, the major impediment to effective treatment is penetration of these therapies across the blood-brain-barrier. Additionally, there are a battery of therapeutic agents that exist to treat patients infected with HIV, however, there are cellular and anatomical reservoirs of the virus that are difficult to eradicate, such as the brain, testes, macrophages and lymphocytes. Viral infection of the brain, for instance, results in a range of HIV-induced neurological complications. The inability to completely eradicate HIV from all of these sites also acts to facilitate viral mutation and ultimately precipitates drug failure. In addition, Glioblastoma multiforme (GBM), also known as grade 4 astrocytoma, is the most common and aggressive type of primary brain tumor, accounting for 52% of all primary brain tumor cases and 20% of all intracranial tumors. One of the largest obstacles to treating GBMs and all brain tumors effectively is the limited ability to deliver chemotherapeutic agents across the blood brain barrier.

Penetration of drugs and other agents into the brain is controlled by the blood brain barrier, a system of capillary endothelial cells that protects the brain from damaging substances in the blood stream. This barrier is comprised of both tight junctions that prevent passage of molecules between cells as well as transport proteins in the endothelial cells which limit uptake. One of the major protein components of the blood-brain-barrier is the transport protein P-glycoprotein (P-gp), which prevents entry of the therapeutic into the brain by actively pumping it back into the blood capillaries. To overcome P-gp's action and get therapeutic amounts of drug into the brain, much larger doses must be given leading to multiple off-target effects and physiological side effects.

Human P-gp is a member of a large superfamily of ATP-dependent proteins known as ABC (ATP-binding cassette) transporters, also including the human ABCG2 and MRP1. P-gp uses the energy of ATP-hydrolysis to pump drug molecules out of cells so that they cannot elicit their therapeutic effects. The P-gp transporter is an integral membrane protein comprised of two homologous halves each containing a cytosolic ATP binding site. ATP binding and hydrolysis are essential for the proper functioning of P-glycoprotein, including drug transport. The drug binding sites in P-gp, on the other hand, are localized to the two transmembrane domains. According to one proposed model, and as shown in FIG. 1, P-gp reduces intracellular drug concentrations by acting as a “hydrophobic vacuum cleaner,” effectively increasing drug efflux and decreasing drug influx by the recognition, and removal of compounds from the membrane before they reach the cytosol to elicit their therapeutic effects.

To probe if P-gp limits the accumulation of CNS active agents in the brain, P-gp-null mice have been studied. Using these mice, P-gp was found to be an important determinant in the brain penetration and pharmacological activity of many drugs (see Schinkel, A. H., Wagenaar, E., Mol, C. A., & van Deemter, L. (1996) P-glycoprotein in the blood-brain barrier of mice influences the brain penetration and pharmacological activity of many drugs, J Clin Invest 97, 2517.). For instance, it has been shown that brain tissue in P-gp-null mice that have been treated with therapies have elevated levels of phenytoin, lamotrigine, carbamazepine, phenobarbital, felbamate, and oxcarbazepine as compared to wild-type mice (see Bebawy, M., and Chetty, M. (2008) Differential phannacological regulation of drug efflux and pharmacoresistant schizophrenia, Bioessays 30, 183 and Potschka, H., and Loscher, W. (2001) In vivo evidence for P-glycoprotein-mediated transport of phenytoin at the blood-brain barrier of rats, Epilepsia 42, 1231). It has also been demonstrated in vivo that pharmacological inhibition of P-gp at the blood brain barrier by valspodar (PSC833) increased the uptake of the anti-epileptic agent phenytoin, a P-gp substrate, in brains (see Potschka, H., and Loscher, W. (2001) In vivo evidence for P-glycoprotein-mediated transport of phenytoin at the blood-brain barrier of rats, Epilepsia 42, 1231). Further confirmation of the role of P-gp efflux at the blood-brain barrier emerged from studies with anti-psychotic agents. Ejsing and co-workers were able to show that the brain-to-serum ratio of risperidone in mdr1a(−/−) knock out mice increased by over 10 times when compared to the ratio in control wild-type mice (see Ejsing, T. B., Pedersen, A. D., & Linnet, K. (2005) P-glycoprotein interaction with risperidone and 9-OH-risperidone studied in vitro, in knock-out mice & in drug-drug interaction experiments, Hum Psycho 20, 493). These data confirm that P-gp appears to play an active role in extruding CNS active agents from the brain, thereby limiting their efficacy.

Anti-HIV therapies that target the viral reverse transcriptase (RT) and protease (PR) are known to be substrates of P-gp, including abacavir, amprenavir, saquinavir, indinavir, and nelfinavir. (see Bachmeier, C. J. et al. “Quanitative assessment of HIV-1 protease inhibitor interactions with drug efflux transporters of the blood-brain barrier” Pharm. Res. (2005) 22, 1259-1268). A comparison of P-gp knock-out mice to their wild-type counterparts has demonstrated a 10 to 40-fold increase in brain penetration for saquinavir and nelfinavir, respectively (see Kim, R. B. et al. “The drug transporter P-glycoprotein limits oral absorption and brain entry of HIV-1 protease inhibitors” J. Clin. Invest. (1998) 101, 289-294). Similarly, the co-administration in mice of the P-gp inhibitor LY335979 and nelfinavir has shown a 25-fold increase in the level of the therapeutic agent in the brain (see Choo, E. F. et al. “Pharmacological inhibition of P-glycoprotein transport enhances the distribution of HIV-1 protease inhibitors into brain and testes” Drug Metab. Dispos. (2000) 28, 655-660). This data indicates that the inhibition of P-gp may be a viable route to eradicate reservoirs of HIV, by allowing anti-HIV agents to accumulate in the cells and organs of interest. Similarly, many of the chemotherapeutic agents used to treat GBMs are, in fact, P-gp substrates, including taxol and gleevec. Recently, Fellner et al demonstrated in vivo and in vitro that co-administration of paclitaxel with the P-gp inhibitor valspodar (PSC833) increased the levels of fluorescently labeled paclitaxel in mouse brains (see Fellner, S. et. al., (2002) Transport of paclitaxel (Taxol) across the blood-brain barrier in vitro and in vivo, J Clin Invest 110, 1309-1318). Furthermore, they demonstrated that co-administration of these two drugs reduced the growth of implanted human glioblastomas by 90% in these mice, whereas treatment with either paclitaxel or valspodar alone had no effect on tumor size.

A striking property of P-gp is its ability to bind and transport a wide range of molecules. The ability of a single protein to interact with such a large variety of molecules suggests the existence of multiple substrate binding sites. Numerous studies have pointed to the existence of at least two spatially distinct substrate binding sites within the transmembrane domain of P-gp that function in transport or regulation of transport (see Shapiro, A. B., and Ling, V. (1997) Positively cooperative sites for drug transport by P-glycoprotein with distinct drug specificities, Eur J Biochem 250, 130). In the recently solved crystal structure of P-gp, it was proposed that the drug binding site formed by the contact between transmembrane helices leads to a large and fluid internal cavity that is able to accommodate the binding of multiple molecules (see Aller, S. G., Yu, J., Ward, A., Weng, Y., Chittaboina, S., Zhuo, R., et al., and Chang, G. (2009) Structure of P-glycoprotein reveals a molecular basis for poly-specific drug binding, Science 323, 1718). This multiplicity of binding pockets within the transport region of P-gp provides a unique opportunity to inhibit P-gp transport with multivalent agents. However, although P-gp inhibition holds great promise for treatment of CNS disease, brain tumors and HIV eradication in the brain and other reservoirs, to date there are only a handful of potent P-gp inhibitors known.

### SUMMARY OF THE INVENTION

The present invention overcomes or ameliorates at least one of the prior art disadvantages discussed above or provides a useful alternative thereto by providing novel inhibitors of P-gp which are dimeric prodrugs of known drugs and are able to revert back to the known therapeutic once inside the reducing or esterase-rich environment of the cell.

According to one aspect of the present invention, a method for inhibiting therapeutic drug resistance within a cell over-expressing a membrane protein is provided. The method comprises synthesizing a dimeric prodrug inhibitor of a monomeric therapeutic agent; administering the dimeric prodrug inhibitor to the membrane protein together with the monomeric therapeutic agent; and occupying at least one substrate binding site of the membrane protein with the synthesized dimeric prodrug to allow the monomeric therapeutic agent to accumulate within the cell. The dimeric prodrug inhibitor contains a crosslinking agent that is adapted to breakdown under reducing conditions within the cytosol of the cell to cause the dimeric prodrug to revert back to a form equivalent to the monomeric therapeutic agent.

According to another aspect of the present invention, a compound of formula (I) is provided:

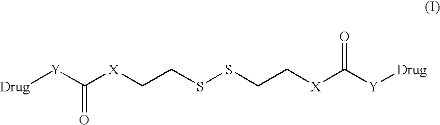

wherein Drug is a monomeric therapeutic agent selected from the group consisting of abacavir, zidovudine, amprenavir, morphine, phenytoin, and venlafaxine; X is CH₂ or O; and Y is O, N or aldehyde/ketone adduct.

Additional embodiments, aspects, and advantages of the present invention will be apparent from the following description.

### BRIEF DESCRIPTION OF THE DRAWINGS

FIG. 1 an exemplary model for P-glycoprotein mechanism of action in accordance with the “hydrophobic vacuum” theory;

FIG. 2 depicts an illustrative generic structure of a dimeric prodrug that is reversible through reduction in accordance with the present invention;

FIG. 3 depicts an illustrative generic structure of dimeric substrates/prodrugs that are reversible through esterase activity in accordance with the present invention;

FIG. 4 depicts disease specific disease dimeric prodrugs in accordance with the present invention that are reversible through reduction for: (a) HIV, (b) Epilepsy, (c) Depression, and (d) Acute Pain;

FIG. 5 depicts disease specific dimeric substrate/prodrugs in accordance with the present invention that are reversible through esterase activity for: (a) HIV, (b) Epilepsy, (c) Cancer, (d) Depression, and (E) Schizophrenia/Psychosis;

FIG. 6 depicts activity of dimeric reducible prodrug inhibitors against the ABC transporters P-gp and ABCG2;

FIG. 7 depicts activity of dimeric esterase-reversible prodrug inhibitors against the ABC transporter P-gp;

FIG. 8 shows a graphical representation of the reversing of cellular Taxol drug resistance with quinine dimerE8 (Q2) inhibitor in accordance with the teachings of the present invention;

FIG. 9 depicts the kinetics of reducing agent-based release of monomer from dimeric prodrugs in accordance with the present invention;

FIG. 10 depicts the ability of dimeric agents to compete for the drug binding sites of P-gp as investigated using an [¹²⁵I]-IAAP photo-affinity labeling assay in accordance with the teachings of the present invention;

FIG. 11 depicts a confocal microscopy image of a rat brain capillary assay taken using fluorescent NBD-cyclosporin A (NBD-CsA) as the substrate;

FIG. 12 depicts a diagrammatic representation of the dimerization of known substrates or drugs by blocking the activity of transporter proteins in accordance with the teaching of the present invention;

FIG. 13a depicts a diagrammatic representation of an administered central nervous system (CNS) active agent that is unable to penetrate the blood-brain barrier;

FIG. 13b depicts a diagrammatic representation of co-administered dimeric P-gp inhibitor (prodrug of CNS agent) with monomeric agent penetrating the blood-brain barrier;

FIG. 13c depicts a diagrammatic representation of co-administered P-gp inhibitor quinine dimer with the monomeric CNS agent penetrating the blood-brain barrier;

FIG. 14 depicts the chemical structures of various exemplary first-line antiepileptic drugs;

FIG. 15a depicts a target hypothesis for a pharmacoresistance model in the brain and having a normal target (a) and a modified drug target not recognized by the administered drug (b);

FIG. 15b depicts a multidrug transporter hypothesis for a pharmacoresistance model in the brain and having a normal expression of multidrug transporters (a) and an overexpression of ABC multidrug transporters in endothelial cells and possible expression in astrocytes and neurons;

FIG. 16 depicts the chemical structure of imatinib mesylate (Gleevec® or STI-571), a P-gb substrate;

FIG. 17 depicts the chemical structures of morphine and galantamine; and

FIG. 18 depicts an exemplary mechanism for the reduction of traceless linkers to convert a dimeric prodrug P-gp inhibitor into a monomeric therapeutic agent in accordance with the present invention;

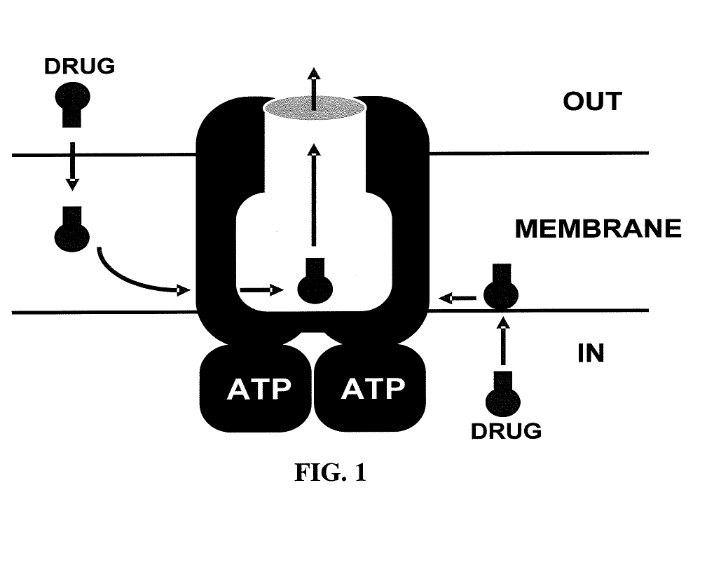

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention. Moreover, while some embodiments incorporating the principles of the present invention have been specifically directed to HIV, cancer and CNS therapeutic applications (e.g., Alzheimer's disease, depression, epilepsy, schizophrenia/psychosis and pain), those skilled in the art should understand and appreciate herein that the present invention may also be directed to other therapeutic areas, such as but not limited to, therapeutic treatments targeted against drug and alcohol addiction and malaria. As such, the present invention is not intended to be limited to the disclosed embodiments. Instead, this application is intended to cover any variations, uses or adaptations of the general principles of the present invention.

In accordance with certain aspects of the present teachings, novel inhibitors of P-gp were designed that are dimeric prodrugs of known therapeutic agents and are particularly useful for inhibiting P-glycoprotein by occupying two substrate binding sites in the transporter to allow monomeric drugs to enter the cell. The dimeric prodrugs that gain entry into the cell are then spontaneously reverted into known approved therapeutics. Data generated in accordance with this aspect of the invention suggest that dimerizing known substrates of P-gp can lead to potent inhibitors of P-gp.

As mentioned above, the present studies and teachings are directed to the eradication of HIV reservoirs and the treatment of brain tumors and CNS diseases. The blood-brain barrier, complete with the multidrug transporter P-glycoprotein, blocks the entry of a number of therapeutics into the brain. As such, the present studies have focused on methods for facilitating brain penetration of anti-HIV, anti-cancer and anti-CNS disease therapeutics by developing novel dimeric prodrugs of P-glycoprotein. To achieve such goals, prodrug dimers of a range of therapies were designed to inhibit P-gp, by simultaneously occupying two substrate binding sites in its transporter region. As will be discussed, this strategy has been successfully used (i.e., dimerizing known P-gp substrates) to obtain potent P-gp inhibitors.

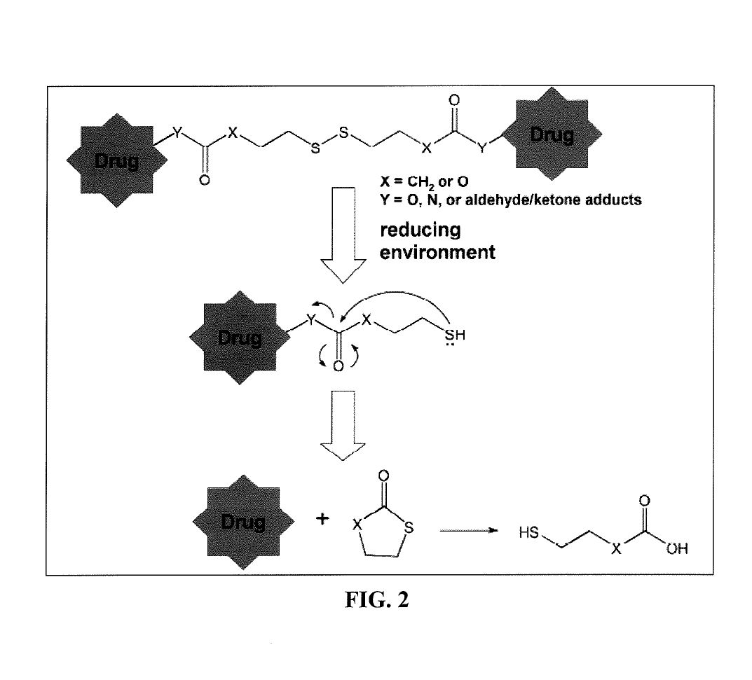

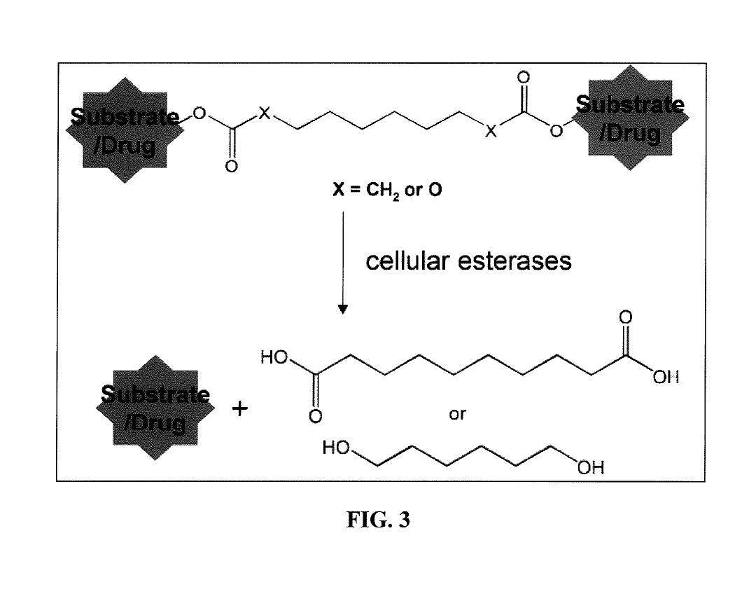

As an additional part of the design for the dimeric prodrugs of the present invention, it was desirable for the dimeric agents to be able to revert back to their corresponding PR inhibitor monomers once cell entry has been accomplished (see FIGS. 2 and 3). To accomplish this, a “traceless” tethering group that is completely removable from the monomers is used. As will be explained in more detail below, two different tethering strategies for dimeric agents are useful in accordance with the teachings of the present invention: (1) dimeric prodrugs that are reversible through reduction (such as shown in FIG. 2), and (2) dimeric substrate/prodrugs that are reversible through esterase activity (such as shown in FIG. 3).

Advantages and improvements of the processes and methods of the present invention are discussed in detail below and demonstrated in the following exemplary examples. These examples are illustrative only and are not intended to limit or preclude other embodiments of the invention.

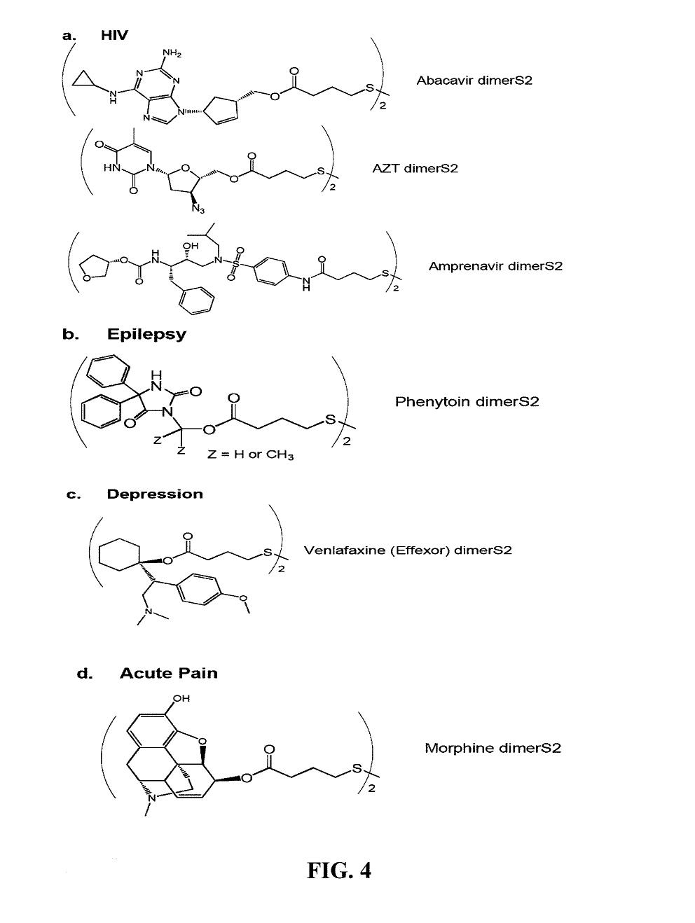

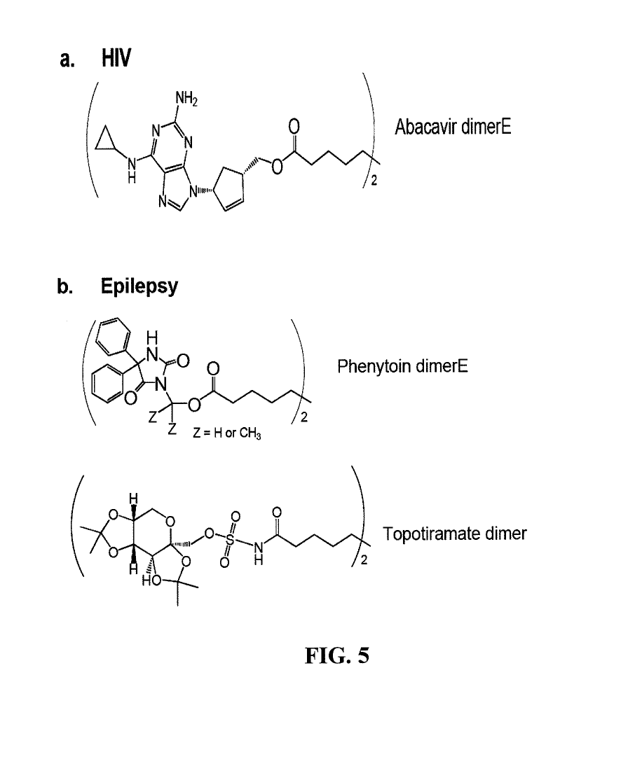

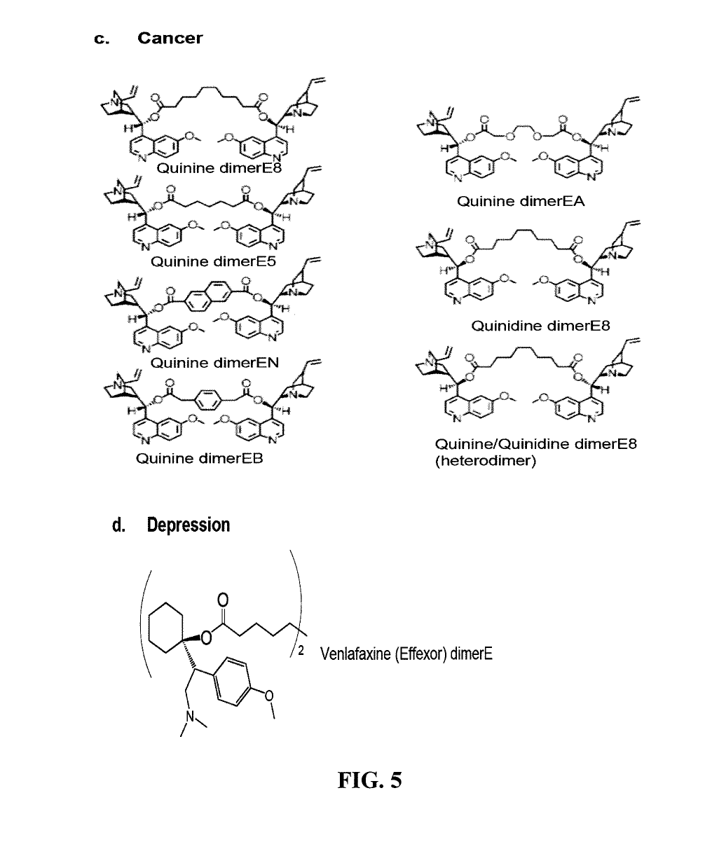

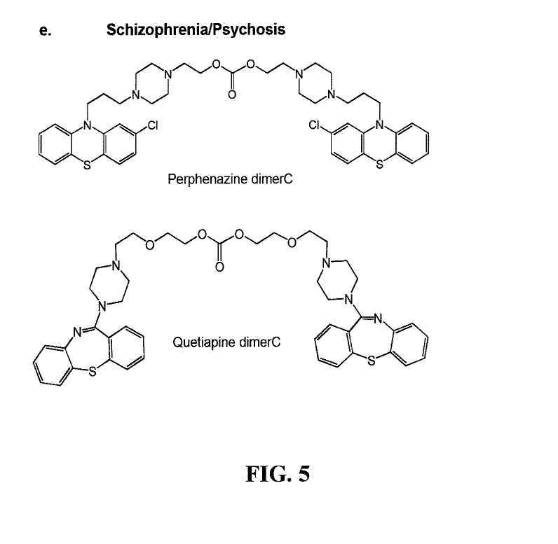

The synthesis of these novel prodrugs proceeded from the corresponding disulfide-bis-carboxylic acid tethers or the bis-carboxylic acid or carbonate tethers and the therapeutic using established procedures (see Vierling, P.; Greiner, J. “Prodrugs of HIV protease inhibitors” Curr. Pharm. Des. (2003) 9, 1755-1770). The dimeric agents synthesized in accordance with the teachings of the present invention are outlined in FIGS. 4 and 5 and grouped by means of tether breakdown and therapeutic areas of use. More particularly, FIG. 4 shows various synthesized prodrug dimers of therapeutic agents that are reversible through reduction, while FIG. 5 shows various synthesized dimeric substrate/prodrugs that are reversible through esterase activity. FIG. 4a shows prodrug dimers that have been synthesized in accordance with the teachings of the present invention for the anti-HIV drugs abacavir, zidovudine (AZT) and amprenavir; FIG. 4b shows a prodrug dimer synthesized for the anti-epilepsy drug phenytoin; FIG. 4c shows a prodrug dimer synthesized for the anti-depression drug venlafaxine (Effexor); and FIG. 4d shows a prodrug dimer synthesized for the anti-actue pain drug morphine. FIG. 5a shows a prodrug dimer synthesized for the anti-HIV drug abacavir; FIG. 5b shows prodrug dimers synthesized for the anti-epilepsy drugs phenytoin, and topotiramate; FIG. 5c shows prodrug dimers synthesized for the anti-cancer drugs quinine and quinidine; FIG. 5d shows a prodrug dimer synthesized for the anti-depression drug venlafaxine (Effexor); and FIG. 5e shows prodrug dimers synthesized for the anti-schizophrenia/psychosis drugs perphenazine and quetiapine.

The inhibition of P-gp transport was evaluated with the prodrug dimers and monomer controls in the human carcinoma MCF7-DX1 cell line. The multidrug resistant subline derived from MCF-7 cells, MCF-7/DX1, was used because it is 200-fold more resistant to doxorubicin (71) and has been shown to express high levels of P-gp by immunoblot analysis (70).

Known fluorescent substrates of P-gp, such as rhodaminel23, were used to evaluate the extent of transport inhibition with all synthetic dimers in the MCF7-DX1 cell lines. Flow cytometry was used to monitor cellular fluorescence with fluorescent substrates, and scintillation counting was used to monitor cellular uptake of radiolabeled daunomycin. The transport inhibition (P-gp or ABCG2) obtained with all synthetic dimeric agents is outlined in FIGS. 6 and 7 and grouped by means of tether breakdown and therapeutic areas.

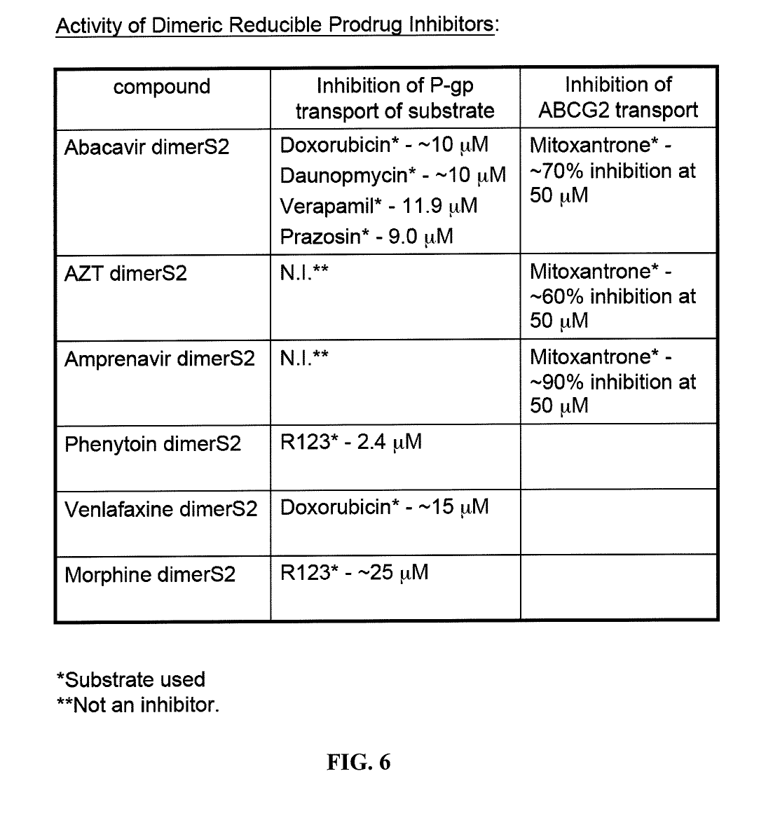

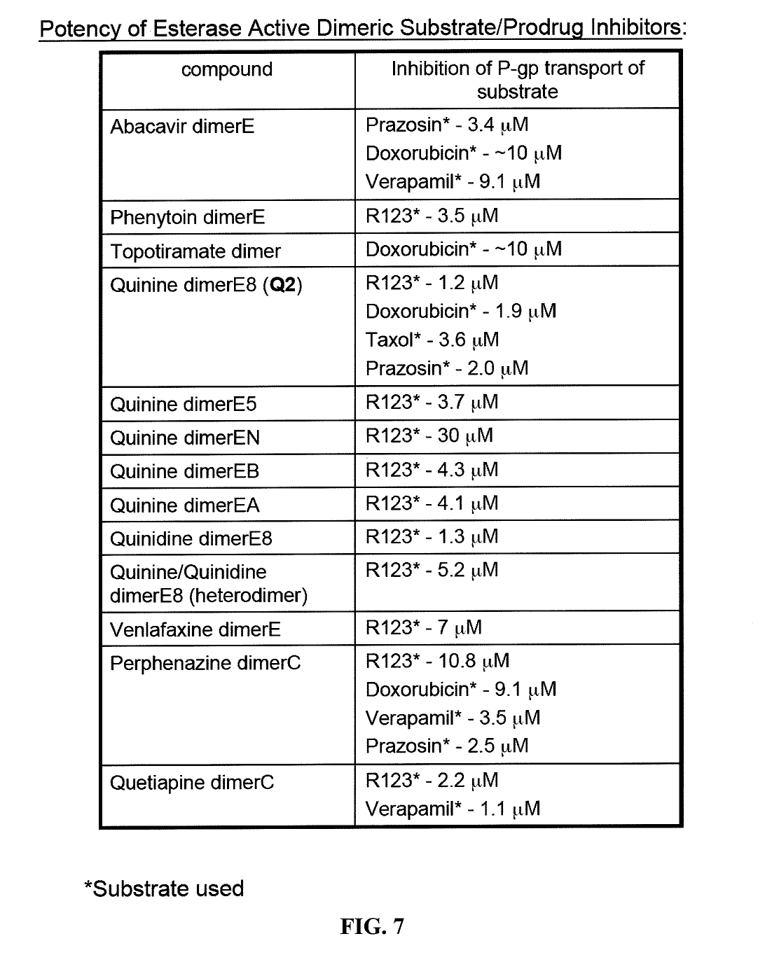

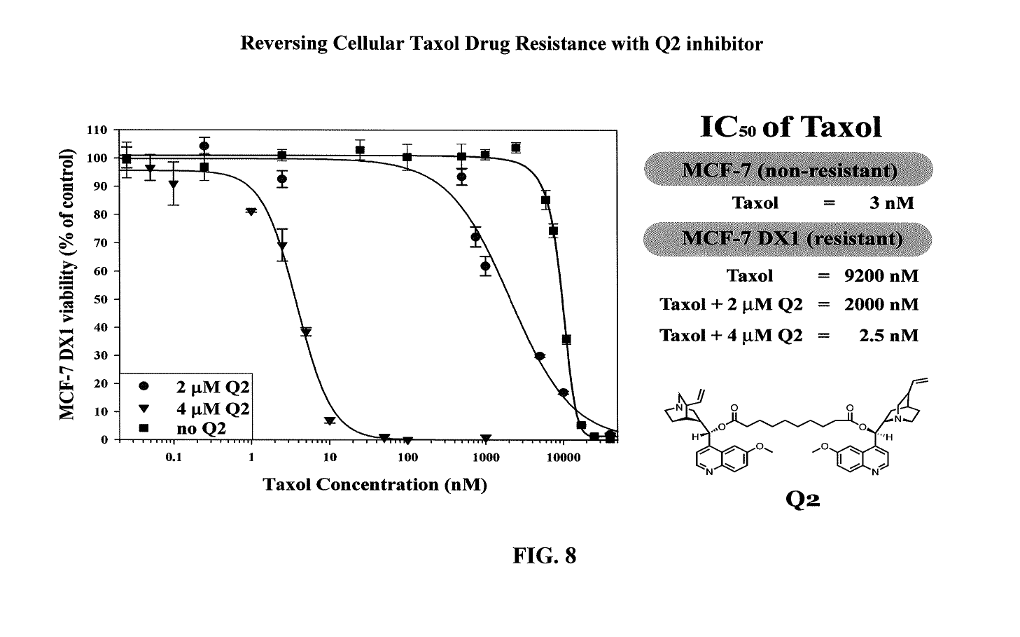

The P-gp-mediated multidrug resistance phenotype of cancer cells is characterized by the ability of cells to evade the cytotoxic effects of drugs that are substrates of P-gp and remain viable even at high extracellular drugs concentrations. One such compound is the cytostatic agent taxol, which is also a known P-gp substrate (Horwitz et al., 1993). Cells lines that over-express P-gp, such as the MCF-7/DX1 cell line, display a 3000-fold decrease in susceptibility to taxol (FIG. 8). The ability of Q2 to reverse the resistance to taxol in these cells was evaluated using the MTT cell viability assay. In these experiments, MCF-7/DX1 cells were co-incubated with taxol and increasing concentrations of Q2 for 72 h. An approximate 4.5-fold decrease in resistance to taxol was observed when cells were incubated with 2 μM of Q2 (FIG. 8), and when taxol was co-incubated with 4 μM of Q2, the potency of taxol was equivalent to that of the non-resistant parental cell line. These results indicated that 4 μM of Q2 is sufficient to fully reverse the taxol-resistant phenotype of these cells. Co-incubation of higher concentrations of Q2 (6 μM) had no additional effect on the IC₅₀ value of taxol. Together, these data demonstrated that low micromolar levels of Q2 led to a 3000-fold increase in potentiation of taxol cytotoxicity, thus completely reversing the resistance phenotype MCF-7/DX1 cells.

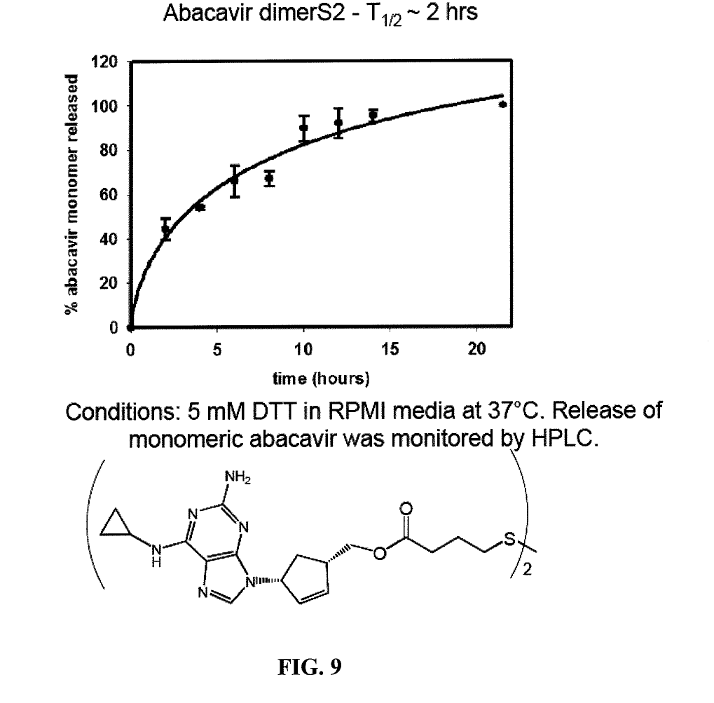

The time course for the release of the traceless linkers from the therapeutic agents was monitored at physiological pH in the presence of the reducing agent dithiothreitol (DTT) (FIG. 9) or pig liver esterase. Since porcine liver esterase is commonly used as a model for enzymatic ester hydrolysis, it was used to determine the stability of the ester bonds in Q2. The reaction was initiated by incubating Q2 (70 μM) in RPMI 1640 cell culture media containing porcine liver esterase (10 units/ml) at 37° C. and hydrolysis was monitored and quantified by reverse phase HPLC. It was observed that Q2 had a half life of approximately 20±2 h. Non-enzymatic hydrolysis was also evaluated by incubating Q2 in cell culture media at 37° C. with no added porcine liver esterase. Under these conditions, Q2 was observed to have high stability in the assay media with a half-life in the order of weeks (21±3 d), an indication that the ester bond is stable to serum and chemical hydrolysis (data not shown). These results suggest that Q2 is likely to remain intact in cell culture assays and should only be degraded following intracellular accumulation.

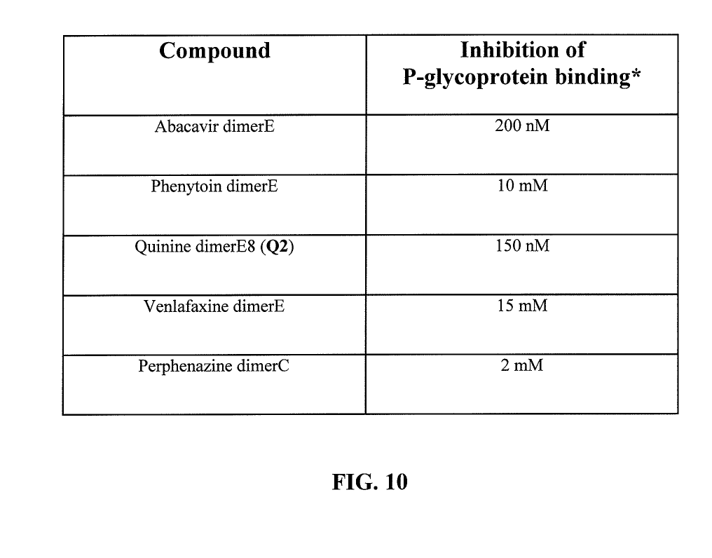

The dimeric prodrugs were designed to interact with the drug binding sites on P-gp and, therefore, they should also compete with other substrates for these binding sites. To verify that this dimeric inhibitors were binding to the drug binding region of P-gp, photo-affinity crosslinking experiments were performed with an I¹²⁵ radiolabeled, azido-analog of prazosin ([¹²⁵I]IAAP) (FIG. 10). Treatment of crude High Five membranes expressing P-gp with [¹²⁵I]IAAP, followed by photocrosslinking, led to the detection of a radiolabeled band on a polyacrylamide gel at the molecular weight corresponding to P-gp, indicating covalent modification of P-gp with IAAP. Co-incubation of P-gp with IAAP and increasing amounts of the dimeric inhibitors was found to inhibit the incorporation of IAAP in a concentration dependent manner (FIG. 10). These findings indicate that the dimeric agents are acting as inhibitors by blocking the drug binding sites within the P-gp transporter region.

P-gp transport inhibition of the fluorescent substrate NBD-cyclosporin A in isolated rat brain capillaries by Q2 was subsequently evaluated. Fluorescence uptake was visualized by confocal microscopy and the amount of accumulated fluorescent compound was quantitated.

Capillaries were isolated according to established protocols (88, 89, 94-96). Briefly, rats were decapitated and the brains put immediately into ice cold Dulbecco's PBS buffer with 5 mM glucose and 1 mM pyruvate. The brains were homogenized and the homogenate mixed with Ficoll at a final concentration of 15% and centrifuged at 5800×g for 10 minutes at 4° C. The pellet was suspended in Dulbecco's PBS with 1% BSA and passed over a glass bead column. The capillaries adhered to the glass and were removed by gentle agitation and subsequently washed three times in BSA-free Dulbecco's PBD. The freshly isolated capillaries were used immediately for transport studies. The capillaries are 5-8 μm in diameter and up to several hundred micrometers in length. It has also been shown that the endothelium in these capillaries presents a barrier to the entry of large molecules (e.g. fluorescein dextrans 10±40 kDa) by diffusion (94).

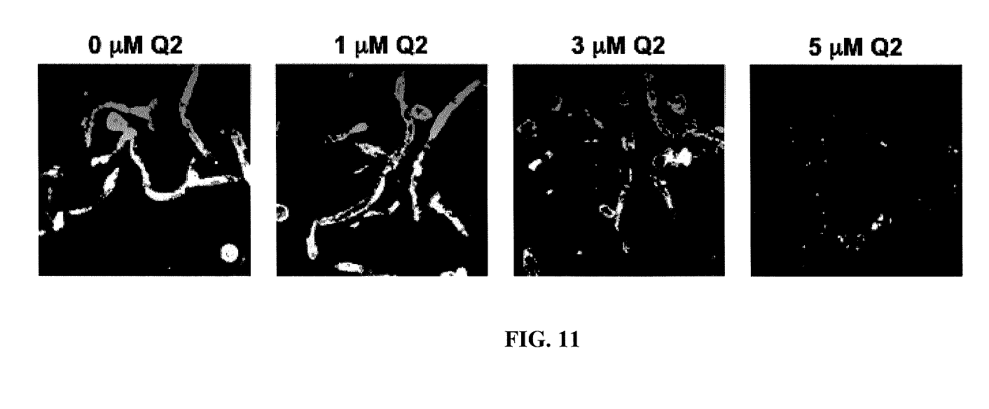

The transport assay was performed as previously described (88, 89, 94-96). Briefly, the isolated capillaries were incubated for 1 hour at room temperature in BSA-free Dulbecco's PBS containing 3 μM of the fluorescent compound NBD-Cyclosporin A in the presence or absence of increasing concentrations of Q2. Confocal images of 10±15 capillaries were acquired using a Zeiss 410 meta laser scanning confocal microscope and luminal fluorescence intensity was measured from stored images using Scion Image software (Scion Corp., Frederick, Md.) (94). In this assay, luminal accumulation of the fluorescent compound is an indication that transport has occurred. Upon inhibition by a P-gp inhibitor, the lumen will accumulate less drug and appear darker. This study showed that Q2 inhibited the P-gp-dependent transport of fluorescent NBD-cyclosporin A into isolated rat brain capillaries in vitro at low micromolar concentrations (FIG. 11).

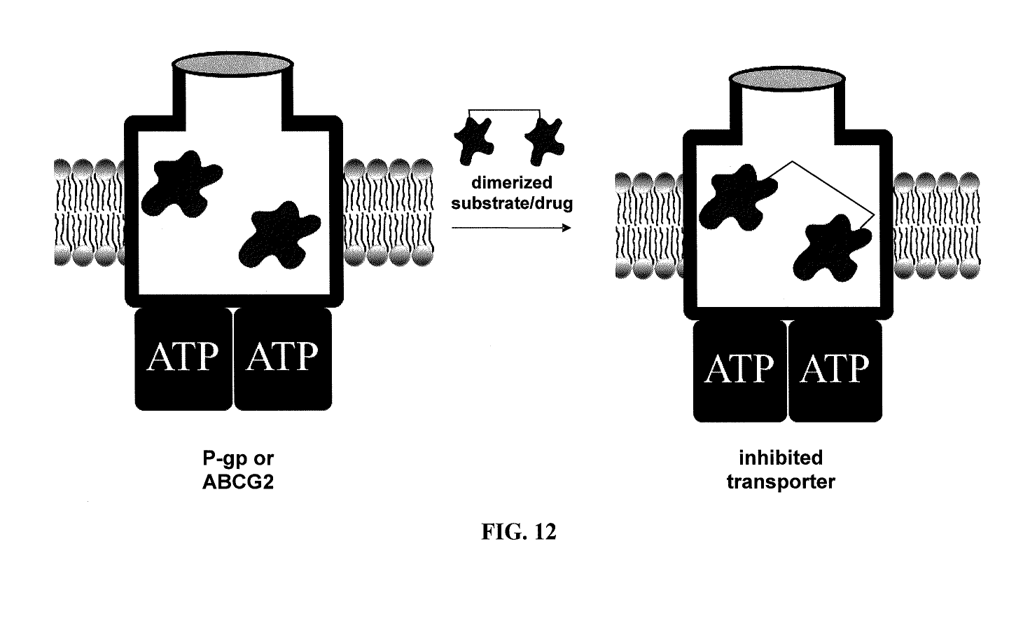

As is generally known and understood, human multidrug resistance ABC transporters (e.g., P-glycoprotein (P-gp) and ABCG2) are membrane proteins that are often over-expressed in a variety of cell lines and endothelial cells of the blood-brain barrier (BBB). As many therapeutic agents are substrates of P-gp and ABCG2, the efficacy of these substrate drugs is often compromised, particularly as the proteins act to exclude drugs from the cells of interest by actively pumping the agent from the cell. However, and in accordance to the teachings of the present invention, the present inventors have devised a strategy whereby the activity of these transporter proteins can be blocked by dimerizing known substrates/drugs (see FIG. 12). More particularly, and as will be explained in great detail below, the co-administration of dimeric inhibitors with monomeric agents can allow for the facile accumulation of the therapeutic agent within cells over-expressing the transporters. Moreover, because the dimeric inhibitor is also a prodrug of the therapeutic agent, the dimeric prodrug inhibitor can breakdown to release the drug, thereby allowing for increased levels of the therapy in cells, such as cancer and BBB cells.

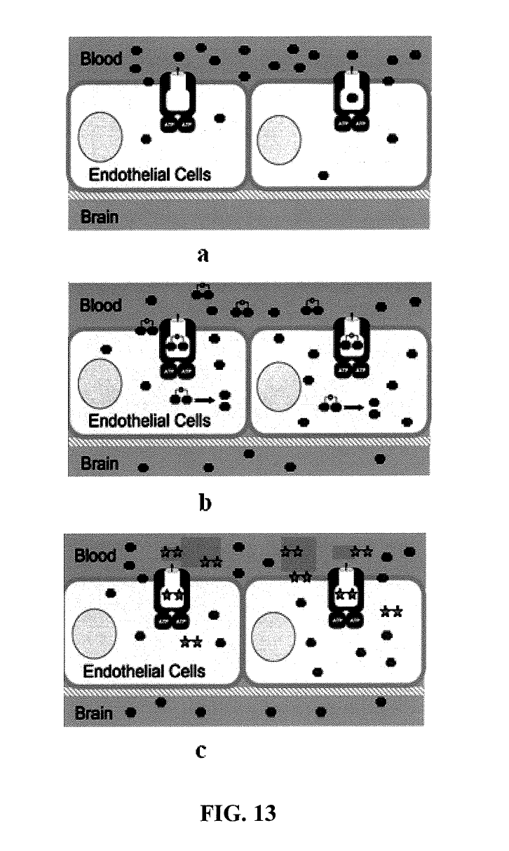

FIG. 13 shows an overall exemplary diagrammatic strategy for dimerizing known substrates or drugs by blocking the activity of transporter proteins in accordance with the general teachings of the present invention. Specifically, FIG. 13a shows the inability of central nervous system active agents (CNS agents—shown in red) to penetrate the BBB and accumulate in the brain. Upon the co-administration of dimeric P-gp inhibitor (13b); however, the BBB is penetrated and the agents can accumulate in the brain. Similarly, and as shown in FIG. 13c, the co-administration of P-gp inhibitor (quinine dimer shown as Q2-green stars) with the monomeric CNS agent (shown in red), also is able to penetrate the BBB and accumulate in the brain.

Epilepsy is a common and serious brain disorder that affects approximately two million people in the United States and 50 million people worldwide (14, 15). Furthermore, according to the World Health Organization (WHO), epilepsy accounts for 1% of the global burden of disease, 80% of which is in the developing world (15). Epilepsy is characterized by the periodic and unpredictable occurrence of seizures, which may be generalized, beginning simultaneously in both hemispheres of the brain, or partial. Partial seizures involve a portion of the brain and most commonly originate in the temporal lobe of the brain (14). Other symptoms of epilepsy can include periods of blank staring, dazed behavior; being unable to talk or communicate for a short time, changes in the way things look, sound, smell or feel and sudden stiffening or falls for no apparent reason.

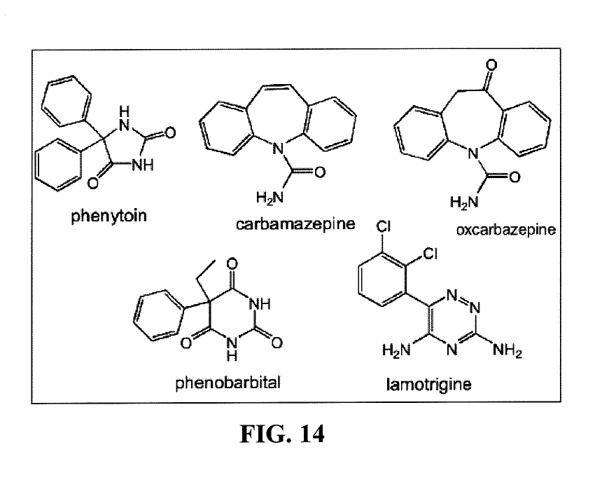

At present, treatment for epilepsy involves the administration of a single first-line antiepileptic drug (AED). These first-line drugs include, but are not limited to, phenytoin (Dilantin®), carbamazepine (Carbatrol®, Tegretol®) and oxcarbazepine (Trileptal®) (14). If these drugs alone are not sufficient to control seizures, a second-line agent is added to the regimen, including lamotrigene (Lamictal) and phenobarbital. The chemical structures of several of these AEDs are shown in FIG. 14. The brain targets for these drugs include voltage-gated Na⁺ channels, GABA neurotransmitter receptors and glutamate receptors (16). Specifically, phenytoin, used for partial and generalized tonic-clonic seizures, acts by producing a voltage and frequency-dependent blockade of voltage-dependent Na⁺ channels and high voltage-activated Ca⁺² channels, thereby stopping sustained firing by neurons during a seizure (16). Lamotrigine (LTG) has a broad spectrum of activity and is useful for treating partial, absence, myoclonic and tonic-clonic seizures (16). LTG is thought to act at voltage-sensitive sodium channels to stabilize neuronal membranes and inhibit the release of excitatory amino acid neurotransmitters. Phenobarbital (PB) is a member of the barbiturate family of compounds and acts by increasing the action of the inhibitory neurotransmitter, GABA in the brain (16). PB may also inhibit the release of glutamate from nerve endings.

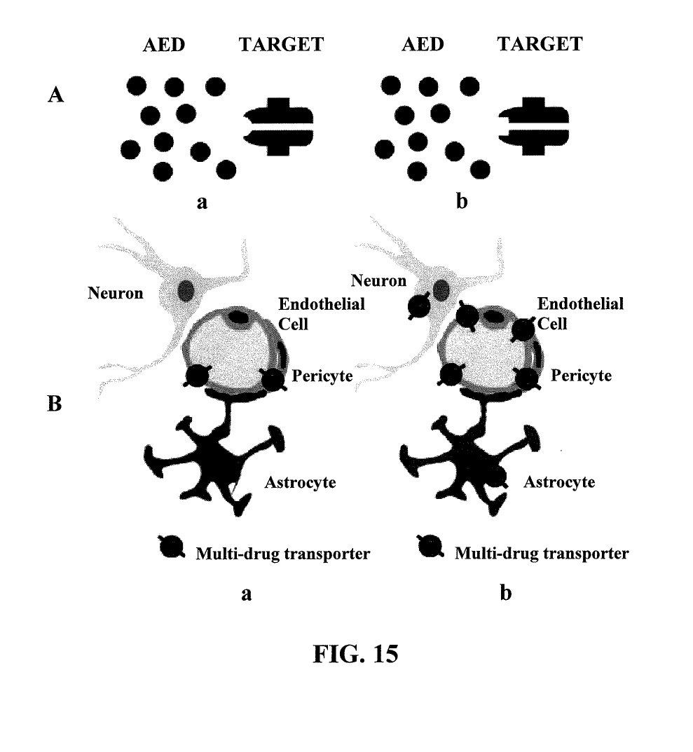

Unfortunately, approximately 30% of epilepsy patients do not respond to many of the first-line AEDs (17, 18). Two complementary and not mutually exclusive hypotheses for drug resistance in epilepsy have emerged over the past several years (see FIG. 15). Firstly, there is the target hypothesis of pharmacoresistance in which changes to the protein target of the drug inside the brain occur, thereby rendering the drug inactive (18). The second hypothesis is called the multidrug transporter hypothesis (19). This hypothesis suggests that the presence of ABC transporters at the blood brain barrier, including P-gp, is responsible for the limited bioavailability of the AEDs at their cellular targets in the brain. This concept was developed in part because the target theory could not adequately explain all of the phenomena associated with AED resistance. First of all, most patients with drug resistant epilepsy are resistant to most, if not all, AEDs regardless of their cellular target (17, 18). Secondly, these agents have little to no structural similarity aside from their hydrophobicity. Third, although adequate plasma levels of the AEDs are achieved in treatments, the brain concentrations remain low.

As described below, three lines of evidence support the hypothesis that P-gp is involved in pharmacoresistance to AEDs. First, the transport of AEDs has been demonstrated in isolated cell systems and isolated brain capillaries. More specifically, in polarized MDCKII dog kidney or LLC-PK1 pig kidney, cell monolayers expressing Pgp, phenytoin and levetiracetam were transported directionally (20). Indirect evidence has shown that phenytoin and lamotrigine are P-gp substrates using a calcein-AM uptake assay with LLC-PK1 pig kidney cell monolayers expressing P-gp (21). Although the focus of this proposal is P glycoprotein, certain AEDs, such as phenytoin and carbamazepine, have also been shown to be substrates for the multidrug resistance associated protein MRP2 (20, 22, 23).

Second, upregulation of P-glycoprotein has been observed in patients and model experimental animal systems and is correlated to a clinical response to AEDs. Increased P-glycoprotein expression in capillary endothelium was observed in brain samples from patients with drug resistant epilepsy (24). Furthermore, a greater than 10-fold increase in mRNA levels of P-gp was also observed in these patients. In a four-month-old patient with seizures resulting from severe tuberous sclerosis that were resistant to phenytoin, phenobarbital, and lorazepam, high levels of P-glycoprotein expression were also observed (25, 26). In some cases, P-gp expression was not limited to the endothelial capillary cells but extended to cells that do not normally express P-gp including astrocytes around blood vessels, hippocampal neurons and glia in rats and humans (27-32) An increased expression of P-gp in the brain capillary endothelial cells was observed in rats with two established models of temporal lobe epilepsy that demonstrated resistance to AED therapy (33, 34). In chemically induced status epilepticus or audiogenic seizures in rats, P-gp expression was found to increase over time following the insult. Correlation of the observed upregulation of P-gp expression to clinical response has also been observed in both humans and rats (24, 33). Taken together, these data suggest a strong and direct relationship between P-gp expression and resistance to AEDs.

Third, the inhibition of P-gp in animal models shows an increase of brain penetration of AEDs. The generation of P-gp knockout mice has also confirmed the important protective pharmacological function of P-gp in the blood brain barrier. In those knock-out mice, the penetration of P-gp substrates, including phenytoin, in the brain was higher than that in the wild-type controls, showing that P-gp restricts the entry of its substrates into the brain (35, 36). Pharmacological inhibition of Pgp also gave promising results in animals and humans. Rats treated with different P-gp inhibitors including verapamil and PSC 833 via a microdialysis probe showed a significant increase in brain phenytoin level than those treated with a control vehicle (1). P-gp-mediated efflux of phenobarbital, lamotrigine and felbamate were also observed in rats using a similar microdialysis experimental approach (37). Two human patients with refractory epilepsy were also co-administered verapamil with AEDs and showed overall improved seizure control, suggesting that inhibition of P-gp allowed for increased brain penetration of the AEDs (38, 39). Using a model of spontaneous seizures in rats, van Vliet et. al (40) co-administered the P-gp inhibitor tariquidar with phenytoin, which resulted in a significant improvement in seizure control compared to phenytoin alone. Most recently, Brandt et al. provided further proof-of-principle of the multidrug transporter hypothesis in refractory epilepsy (41). The investigators used a rat model of temporal lobe epilepsy and selected drug-resistant and drug-sensitive subgroups of epileptic rats using phenobarbital. A group of drug-resistant rats were subsequently treated with phenobarbital in conjunction with the P-gp inhibitor tariquidar. Remarkably, this co-administration resulted in full restoration of the activity of phenobarbital. Together, these data suggest that therapies involving the inhibition of P-gp in conjunction with the administration of AEDs may be a promising treatment strategy for drug resistant epilepsy patients.

Alzheimer's disease (AD) is a progressive disorder that results in loss of cognition and memory. Estimates from the 2000 census indicate that approximately 4.5 million Americans have Alzheimer's disease, a number that has doubled since 1980 (42). By 2050, the number of affected individuals could range from 11.3 million to 16 million (42). Unfortunately, although there are five drugs approved for AD, none are truly effective treatments for the disease. However, efforts to develop different and more potent treatments are ongoing (43).

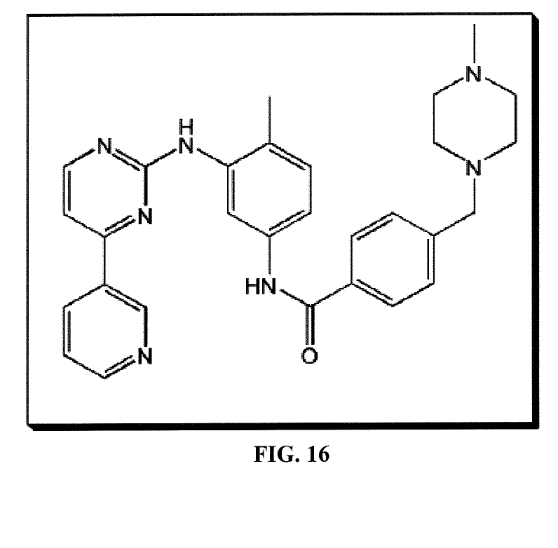

Amyloid β (Aβ) peptide fibrils accumulate in the brain of Alzheimer's patients, a phenomenon that may contribute to the pathogenesis of AD (43). One particularly intriguing avenue of research has been in reducing Aβproduction. Aβ is produced from the proteolytic processing of amyloid precursor protein (APP) by two proteases, β- and γ-secretase (43). Several inhibitors of γ-secretase directed against the protease substrate site are in clinical trials. However, these compounds are not selective and other important proteins, such as the Notch proteins, are also not processed, resulting in deleterious and toxic effects (44). An important discovery was made recently that demonstrated that the protein tyrosine kinase inhibitor imatinib mesylate (Gleevec or STI-571) (FIG. 16) reduced the production of Aβ but did not inhibit Notch cleavage (45, 46). It was subsequently determined that γ-secretase contains an allosteric nucleotide binding domain and that binding of certain compounds, including Gleevec®, prevents APP cleavage but leaves Notch processing intact. Gleevec® has also been shown to potently inhibit the formation of Aβ in rat primary neuronal cultures and in vivo in guinea pig brain (46). Gleevec®, however, is a substrate for both P-glycoprotein and ABCG2 at the blood brain barrier (47-50), a fact that makes its use for AD treatment difficult. Importantly, it has been shown that pharmacological inhibition of P-gp and ABCG2 increased the brain penetration of Gleevec® significantly (48), suggesting that this combination therapy may improve the delivery of Gleevec® to the brain and limit Aβ formation.

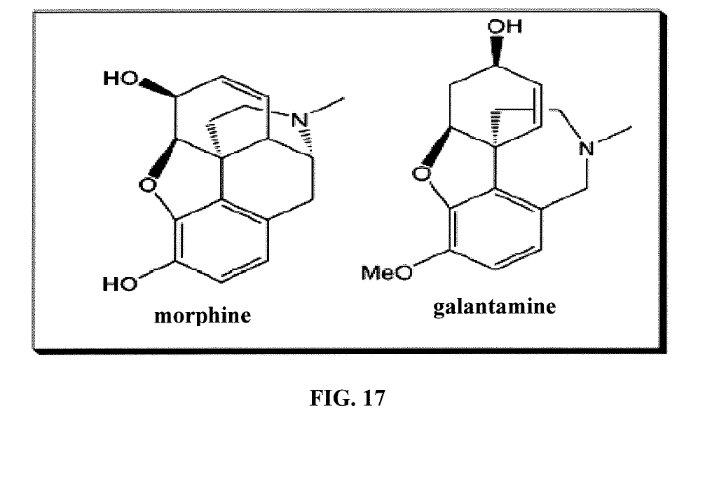

Galantamine (Reminyl®) (FIG. 17) is one of the FDA approved acetylcholinesterase inhibitors used for the treatment of Aβ (51, 52) and has been shown to improve cognition and alleviate symptoms. AD is thought to be in part due to the loss of cholinergic neurons in the basal forebrain (53, 54). Therefore, by inhibiting acetylcholinesterase, galantamine increases the amount of acetylycholine in central synapses. Galantamine also is an allosteric activating ligand of nicotinic acetylcholine receptors. Although it is not known whether galantamine itself is a P-gp substrate, it is very similar chemically to morphine (FIG. 17), a well-known P-gp substrate at the blood brain barrier (55-58). It is hypothesized that P-gp limits the amount of galantamine entering the brain and thus lowers its effectiveness.

Co-administration of the dimeric prodrugs and the monomeric drugs, therefore, would allow for accumulation of the therapeutic drug within the brain via two pathways: enhanced entry of monomeric drug through inhibition of P-gp at the BBB, and breakdown of the dimeric P-gp inhibitors within endothelial cells at the BBB to provide more of the monomeric drug (see FIG. 13). The dimeric prodrugs of phenytoin, phenobarbital and lamotrigine, therefore, will serve two purposes: (1) inhibit P glycoprotein at the BBB by occupying two substrates binding sites in the transporter and (2) prodrug dimers that gain entry into the endothelial cells at the BBB would revert to their monomeric forms in the reducing environment of the cytosol.

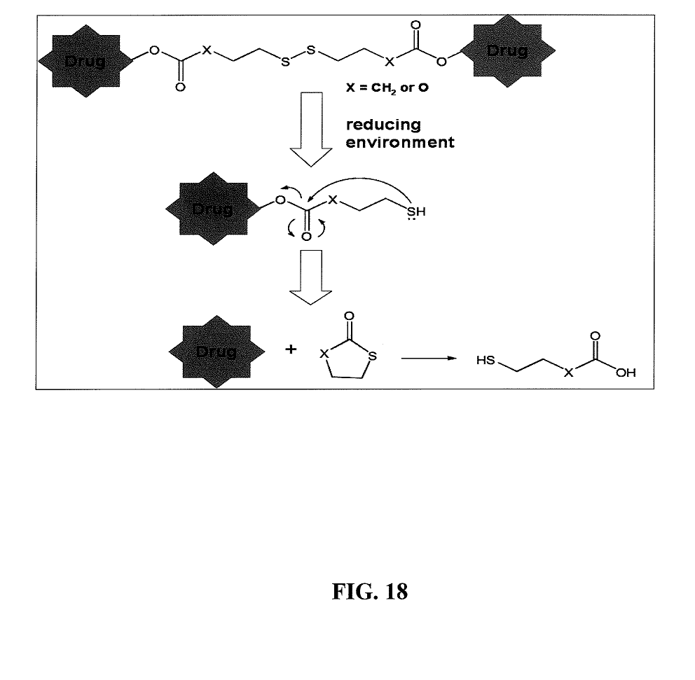

Specifically, it is proposed to use a tether containing an internal disulfide with carbonate, ester or carbamate linkages to the monomeric drugs (FIG. 18). Under reducing conditions, such as those that exist in the cytosol, the disulfide linkage would reduce to the thiol. This thiol moiety would then act as a nucleophile, resulting in cleavage of the link to the drug and regeneration of the therapeutic agent with release of the tether as a non-toxic by-product.

While an exemplary embodiment incorporating the principles of the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

The following references are incorporated herein by reference in their entirety:
1. Potschka, H., and Loscher, W. (2001) In vivo evidence for P-glycoprotein-mediated transport of phenytoin at the blood-brain barrier of rats, Epilepsia 42, 1231-1240.
2. Cordon-Cardo, C., O'Brien, J. P., Casals, D., Rittman-Grauer, L., Biedler, J. L., Melamed, M. R., and Bertino, J. R. (1989) Multidrug-resistance gene (P glycoprotein) is expressed by endothelial cells at blood-brain barrier sites, Proc Natl Acad Sci USA 86, 695-698.
3. Loscher, W., and Potschka, H. (2002) Role of multidrug transporters in pharmacoresistance to antiepileptic drugs, The Journal of pharmacology and experimental therapeutics 301, 7-14.
4. Schinkel, A. H. (1999) P-Glycoprotein, a gatekeeper in the blood-brain barrier, Adv Drug Deliv Rev 36, 179-194.
5. Gottesman, M. M., Hrycyna, C. A., Schoenlein, P. V., Germann, U. A., and Pastan, I. (1995) Genetic analysis of the multidrug transporter, Annu Rev Genet 29, 607-649.
6. Gottesman, M. M., and Pastan, I. (1993) Biochemistry of multidrug resistance mediated by the multidrug transporter, Annu. Rev. Biochem. 62, 385-427.
7. Higgins, C. F. (1992) ABC transporters: from microorganisms to man, Annu Rev Cell Biol 8, 67-113.
8. Hrycyna, C. A. (2001) Molecular genetic analysis and biochemical characterization of mammalian P-glycoproteins involved in multidrug resistance, Semin Cell Dev Biol 12, 247-256.
9. Bruggemann, E. P., Currier, S. J., Gottesman, M. M., and Pastan, I. (1992) Characterization of the azidopine and vinblastine binding site of P-glycoprotein, J. Biol. Chem. 267, 21020-21026.
10. Bruggemann, E. P., Germann, U. A., Gottesman, M. M., and Pastan, I. (1989) Two different regions of P-glycoprotein are photoaffinity labeled by azidopine, J. Biol. Chem. 264, 15483-15488.
11. Greenberger, L. M. (1993) Major photoaffinity drug labeling sites for iodoaryl azidoprazosin in P-Glycoprotein are within, or immediately C-Terminal to, transmembrane domain-6 and domain-12, J. Biol. Chem. 268, 11417-11425.
12. Horio, M., Gottesman, M. M., and Pastan, I. (1988) ATP-dependent transport of vinblastine in vesicles from human multidrug-resistant cells, Proc. Natl. Acad. Sci. USA 85, 3580-3584.
13. Raviv, Y., Pollard, H. B., Bruggemann, E. P., Pastan, I., and Gottesman, M. M. (1990) Photosensitized labeling of a functional multidrug transporter in living drug resistant tumor cells, J. Biol. Chem. 265, 3975-3980.
14. Browne, T. R., and Holmes, G. L. (2001) Epilepsy, The New England Journal of Medicine 344, 1145-1151.
15. WHO. (2005) Atlas: Epilepsy care in the world 2005, (Organization, W. H., Ed.).
16. Kwan, P., Sills, G. J., and Brodie, M. J. (2001) The mechanisms of action of commonly used antiepileptic drugs, Pharmacology & therapeutics 90, 21-34.
17. Regesta, G., and Tanganelli, P. (1999) Clinical aspects and biological bases of drug-resistant epilepsies, Epilepsy research 34, 109-122.
18. Remy, S., and Beck, H. (2006) Molecular and cellular mechanisms of pharmacoresistance in epilepsy, Brain 129, 18-35.
19. Kwan, P., and Brodie, M. J. (2005) Potential role of drug transporters in the pathogenesis of medically intractable epilepsy, Epilepsia 46, 224-235.
20. Baltes, S., Gastens, A. M., Fedrowitz, M., Potschka, H., Kaever, V., and Loscher, W. (2006) Differences in the transport of the antiepileptic drugs phenytoin, levetiracetam and carbamazepine by human and mouse P-glycoprotein, Neuropharmacology.
21. Weiss, J., Kerpen, C. J., Lindenmaier, H., Dormann, S. M., and Haefeli, W. E. (2003) Interaction of antiepileptic drugs with human P-glycoprotein in vitro, The Journal of pharmacology and experimental therapeutics 307, 262-267.
22. Potschka, H., Fedrowitz, M., and Loscher, W. (2003) Brain access and anticonvulsant efficacy of carbamazepine, lamotrigine, and felbamate in ABCC2/MRP2-deficient TR-rats, Epilepsia 44, 1479-1486.
23. Potschka, H., Fedrowitz, M., and Loscher, W. (2003) Multidrug resistance protein MRP2 contributes to blood-brain barrier function and restricts antiepileptic drug activity, The Journal of pharmacology and experimental therapeutics 306, 124-131.
24. Tishler, D. M., Weinberg, K. I., Hinton, D. R., Barbaro, N., Annett, G. M., and Raffel, C. (1995) MDR1 gene expression in brain of patients with medically intractable epilepsy, Epilepsia 36, 1-6.
25. Lazarowski, A., Lubieniecki, F., Camarero, S., Pomata, H., Bartuluchi, M., Sevlever, G., and Taratuto, A. L. (2004) Multidrug resistance proteins in tuberous sclerosis and refractory epilepsy, Pediatric neurology 30, 102-106.
26. Lazarowski, A., Sevlever, G., Taratuto, A., Massaro, M., and Rabinowicz, A. (1999) Tuberous sclerosis associated with MDR1 gene expression and drug-resistant epilepsy, Pediatric neurology 21, 731-734.
27. Aronica, E., Gorter, J. A., Jansen, G. H., van Veelen, C. W., van Rijen, P. C., Leenstra, S., Ramkema, M., Scheffer, G. L., Scheper, R. J., and Troost, D. (2003) Expression and cellular distribution of multidrug transporter proteins in two major causes of medically intractable epilepsy: focal cortical dysplasia and glioneuronal tumors, Neuroscience 118, 417 429.
28. Lu, Y., Yan, Y., and Wang, X. F. (2004) Antiepileptic drug-induced multidrug resistance Pglycoprotein overexpression in astrocytes cultured from rat brains, Chinese medical journal 117, 1682-1686.
29. Sisodiya, S. M. (2003) Mechanisms of antiepileptic drug resistance, Current opinion in neurology 16, 197-201.
30. Sisodiya, S. M., Heffernan, J., and Squier, M. V. (1999) Over expression of P-glycoprotein in malformations of cortical development, Neuroreport 10, 3437-3441.
31. Sisodiya, S. M., Lin, W. R., Harding, B. N., Squier, M. V., and Thom, M. (2002) Drug resistance in epilepsy: expression of drug resistance proteins in common causes of refractory epilepsy, Brain 125, 22-31.
32. Sisodiya, S. M., Lint, W. R., Harding, B. N., Squier, M. V., and Thom, M. (2002) Drug resistance in epilepsy: human epilepsy, Novartis Foundation symposium 243, 167-174; discussion 174-169, 180-165.
33. Volk, H. A., and Loscher, W. (2005) Multidrug resistance in epilepsy: rats with drug resistant seizures exhibit enhanced brain expression of P-glycoprotein compared with rats with drug-responsive seizures, Brain 128, 1358-1368.
34. Volk, H. A., Potschka, H., and Loscher, W. (2004) Increased expression of the multidrug transporter P-glycoprotein in limbic brain regions after amygdala-kindled seizures in rats, Epilepsy research 58, 67-79.
35. Schinkel, A. H., Mayer, U., Wagenaar, E., Mol, C. A., van Deemter, L., Smit, J. J., van der Valk, M. A., Voordouw, A. C., Spits, H., van Tellingen, O., Zijlmans, J. M., Fibbe, W. E., and Borst, P. (1997) Normal viability and altered pharmacokinetics in mice lacking mdr1 type (drug-transporting) P-glycoproteins, Proc Natl Acad Sci USA 94, 4028-4033.
36. Schinkel, A. H., Wagenaar, E., Mol, C. A., and van Deemter, L. (1996) P-glycoprotein in the blood-brain barrier of mice influences the brain penetration and pharmacological activity of many drugs, J Clin Invest 97, 2517-2524.
37. Potschka, H., Fedrowitz, M., and Loscher, W. (2002) P-Glycoprotein-mediated efflux of phenobarbital, lamotrigine, and felbamate at the blood-brain barrier: evidence from microdialysis experiments in rats, Neuroscience letters 327, 173-176.
38. Iannetti, P., Spalice, A., and Parisi, P. (2005) Calcium-channel blocker verapamil administration in prolonged and refractory status epilepticus, Epilepsia 46, 967-969.
39. Summers, M. A., Moore, J. L., and McAuley, J. W. (2004) Use of verapamil as a potential Pglycoprotein inhibitor in a patient with refractory epilepsy, The Annals of pharmacotherapy 38, 1631-1634.
40. van Vliet, E. A., van Schaik, R., Edelbroek, P. M., Redeker, S., Aronica, E., Wadman, W. J., Marchi, N., Vezzani, A., and Gorter, J. A. (2006) Inhibition of the multidrug transporter Pglycoprotein improves seizure control in phenytoin-treated chronic epileptic rats, Epilepsia 47, 672-680.
41. Brandt, C., Bethmann, K., Gastens, A. M., and Loscher, W. (2006) The multidrug transporter hypothesis of drug resistance in epilepsy: Proof-of-principle in a rat model of temporal lobe epilepsy, Neurobiology of disease 24, 202-211.
42. Hebert, L. E., Scherr, P. A., Bienias, J. L., Bennett, D. A., and Evans, D. A. (2003) Alzheimer disease in the US population: prevalence estimates using the 2000 census, Archives of neurology 60, 1119-1122.
43. Roberson, E. D., and Mucke, L. (2006) 100 years and counting: prospects for defeating Alzheimer's disease, Science 314, 781-784.
44. Wolfe, M. S. (2006) The gamma-secretase complex: membrane-embedded proteolytic ensemble, Biochemistry 45, 7931-7939.
45. Fraering, P. C., Ye, W., LaVoie, M. J., Ostaszewski, B. L., Selkoe, D. J., and Wolfe, M. S. (2005) gamma-Secretase substrate selectivity can be modulated directly via interaction with a nucleotide-binding site, The Journal of biological chemistry 280, 41987-41996.
46. Netzer, W. J., Dou, F., Cai, D., Veach, D., Jean, S., Li, Y., Bornmann, W. G., Clarkson, B., Xu, H., and Greengard, P. (2003) Gleevec inhibits beta-amyloid production but not Notch cleavage, Proc Natl Acad Sci USA 100, 12444-12449.
47. Breedveld, P., Beijnen, J. H., and Schellens, J. H. (2006) Use of P-glycoprotein and BCRP inhibitors to improve oral bioavailability and CNS penetration of anticancer drugs, Trends in pharmacological sciences 27, 17-24.
48. Breedveld, P., Pluim, D., Cipriani, G., Wieling a, P., van Tellingen, O., Schinkel, A. H., and Schellens, J. H. (2005) The effect of Bcrp1 (Abcg2) on the in vivo pharmacokinetics and brain penetration of imatinib mesylate (Gleevec: implications for the use of breast cancer resistance protein and P-glycoprotein inhibitors to enable the brain penetration of imatinib in patients, Cancer research 65, 2577-2582.
49. Dai, H., Marbach, P., Lemaire, M., Hayes, M., and Elmquist, W. F. (2003) Distribution of STI-571 to the brain is limited by P-glycoprotein-mediated efflux, The Journal of pharmacology and experimental therapeutics 304, 1085-1092.
50. Ozvegy-Laczka, C., Hegedus, T., Varady, G., Ujhelly, O., Schuetz, J. D., Varadi, A., Keri, G., Orfi, L., Nemet, K., and Sarkadi, B. (2004) High-affinity interaction of tyrosine kinase inhibitors with the ABCG2 multidrug transporter, Molecular pharmacology 65, 1485-1495.
51. Grutzendler, J., and Morris, J. C. (2001) Cholinesterase inhibitors for Alzheimer's disease, Drugs 61, 41-52.
52. Thompson, C. A. (2001) FDA approves galantamine for Alzheimer's disease, Am J Health Syst Pharm 58, 649.
53. Bartus, R. T., Dean, R. L., 3rd, Beer, B., and Lippa, A. S. (1982) The cholinergic hypothesis of geriatric memory dysfunction, Science 217, 408-414.
54. Whitehouse, P. J., Price, D. L., Clark, A. W., Coyle, J. T., and DeLong, M. R. (1981) Alzheimer disease: evidence for selective loss of cholinergic neurons in the nucleus basalis, Annals of neurology 10, 122-126.
55. Cisternino, S., Rousselle, C., Debray, M., and Scherrmann, J. M. (2004) In situ transport of vinblastine and selected P-glycoprotein substrates: implications for drug-drug interactions at the mouse blood-brain barrier, Pharmaceutical research 21, 1382-1389.
56. Huwyler, J., Drewe, J., Gutmann, H., Thole, M., and Fricker, G. (1998) Modulation of morphine-6-glucuronide penetration into the brain by P-glycoprotein, International journal of clinical pharmacology and therapeutics 36, 69-70.
57. Huwyler, J., Drewe, J., Klusemann, C., and Fricker, G. (1996) Evidence for P glycoproteinmodulated penetration of morphine-6-glucuronide into brain capillary endothelium, British journal of pharmacology 118, 1879-1885.
58. Zong, J., and Pollack, G. M. (2000) Morphine antinociception is enhanced in mdr1a genedeficient mice, Pharmaceutical research 17, 749-753.
59. Dey, S., Ramachandra, M., Pastan, I., Gottesman, M. M., and Ambudkar, S. V. (1997) Evidence for two nonidentical drug-interaction sites in the human P-glycoprotein, Proc. Natl. Acad. Sci. USA 94, 10594-10599.
60. Loo, T. W., Bartlett, M. C., and Clarke, D. M. (2003) Simultaneous binding of two different drugs in the binding pocket of the human multidrug resistance P-glycoprotein, The Journal of biological chemistry 278, 39706-39710.
61. Loo, T. W., Bartlett, M. C., and Clarke, D. M. (2003) Methanethiosulfonate derivatives of rhodamine and verapamil activate human P-glycoprotein at different sites, The Journal of biological chemistry 278, 50136-50141.
62. Martin, C., Berridge, G., Higgins, C. F., Mistry, P., Charlton, P., and Callaghan, R. (2000) Communication between multiple drug binding sites on P-glycoprotein, Molecular pharmacology 58, 624-632.
63. Shapiro, A. B., and Ling, V. (1997) Positively cooperative sites for drug transport by Pglycoprotein with distinct drug specificities, Eur J Biochem 250, 130-137.
64. Wang, E. J., Casciano, C. N., Clement, R. P., and Johnson, W. W. (2000) Two transport binding sites of P-glycoprotein are unequal yet contingent: initial rate kinetic analysis by ATP hydrolysis demonstrates intersite dependence, Biochimica et biophysica acta 1481, 63 74.
65. Sauna, Z. E., Andrus, M. B., Turner, T. M., and Ambudkar, S. V. (2004) Biochemical basis of polyvalency as a strategy for enhancing the efficacy of P-glycoprotein (ABCB1) modulators: stipiamide homodimers separated with defined-length spacers reverse drug efflux with greater efficacy, Biochemistry 43, 2262-2271.
66. Pires, M. M., Hrycyna, C. A., and Chmielewski, J. (2006) Bivalent probes of the human multidrug transporter P-glycoprotein, Biochemistry 45, 11695-11702.
67. Ambudkar, S. V., Kimchi-Sarfaty, C., Sauna, Z. E., and Gottesman, M. M. (2003) Pglycoprotein: from genomics to mechanism, Oncogene 22, 7468-7485.
68. Robert, J., and Jarry, C. (2003) Multidrug resistance reversal agents, Journal of medicinal chemistry 46, 4805-4817.
69. Wang, R. B., Kuo, C. L., Lien, L. L., and Lien, E. J. (2003) Structure-activity relationship: analyses of p-glycoprotein substrates and inhibitors, Journal of clinical pharmacy and therapeutics 28, 203-228.
70. Ejendal, K. F., and Hrycyna, C. A. (2005) Differential sensitivities of the human ATP binding cassette transporters ABCG2 and P-glycoprotein to cyclosporin A, Molecular pharmacology 67, 902-911.
71. Fairchild, C. R., Ivy, S. P., Kao-Shan, C. S., Whang-Peng, J., Rosen, N., Israel, M. A., Melera, P. W., Cowan, K. H., and Goldsmith, M. E. (1987) Isolation of amplified and overexpressed DNA sequences from adriamycin-resistant human breast cancer cells, Cancer research 47, 5141-5148.
72. Leonessa, F., Jacobson, M., Boyle, B., Lippman, J., McGarvey, M., and Clarke, R. (1994) Effect of tamoxifen on the multidrug-resistant phenotype in human breast cancer cells: isobologram, drug accumulation, and M(r) 170,000 glycoprotein (gp170) binding studies, Cancer research 54, 441-447.
73. Borgnia, M. J., Eytan, G. D., and Assaraf, Y. G. (1996) Competition of hydrophobic peptides, cytotoxic drugs, and chemosensitizers on a common P-glycoprotein pharmacophore as revealed by its ATPase activity, The Journal of biological chemistry 271, 3163-3171.
74. Hayeshi, R., Masimirembwa, C., Mukanganyama, S., and Ungell, A. L. (2006) The potential inhibitory effect of antiparasitic drugs and natural products on P-glycoprotein mediated efflux, Eur J Pharm Sci 29, 70-81.
75. Damen, E. W., Nevalainen, T. J., van den Bergh, T. J., de Groot, F. M., and Scheeren, H. W. (2002) Synthesis of novel paclitaxel prodrugs designed for bioreductive activation in hypoxic tumour tissue, Bioorganic & medicinal chemistry 10, 71-77.
76. Henne, W. A., Doorneweerd, D. D., Hilgenbrink, A. R., Kularatne, S. A., and Low, P. S. (2006) Synthesis and activity of a folate peptide camptothecin prodrug, Bioorganic & medicinal chemistry letters 16, 5350-5355.
77. Jones, L. R., Goun, E. A., Shinde, R., Rothbard, J. B., Contag, C. H., and Wender, P. A. (2006) Releasable luciferin-transporter conjugates: tools for the real-time analysis of cellular uptake and release, Journal of the American Chemical Society 128, 6526-6527.
78. Stella, V. J. (1996) A case for prodrugs: Fosphenytoin, Advanced Drug Delivery Reviews 19, 311-330.
79. Bosch, J., Roca, T., Domenech, J., and Suriol, M. (1999) Synthesis of water-soluble phenytoin prodrugs, Bioorganic & medicinal chemistry letters 9, 1859-1862.
80. Dalpozzo, A., and Acquasaliente, M. (1992) New Highly Water-Soluble Phenytoin Prodrugs, International Journal of Pharmaceutics 81, 263-265.
81. Jing, B., Janout, V., and Regen, S. L. (2003) Fully detachable molecular umbrellas as peptide delivery agents, Bioconjugate chemistry 14, 1191-1196.
82. Li, J., Zheng, L. M., King, I., Doyle, T. W., and Chen, S. H. (2001) Syntheses and antitumor activities of potent inhibitors of ribonucleotide reductase: 3-amino-4 methylpyridine-2-carboxaldehyde-thiosemicarba-zone (3-AMP), 3-amino-pyridine-2 carboxaldehydethiosemicarbazone (3-AP) and its water-soluble prodrugs, Current medicinal chemistry 8, 121-133.
83. Senter, P. D., Pearce, W. E., and Greenfield, R. S. (1990) Development of a Drug-Release Strategy Based on the Reductive Fragmentation of Benzyl Carbamate Disulfides, Journal of Organic Chemistry 55, 2975-2978.
84. Hrycyna, C. A., Ramachandra, M., Pastan, I., and Gottesman, M. M. (1998) Functional expression of human P-glycoprotein from plasmids using vaccinia virus-bacteriophage T7 RNA polymerase system, Methods in enzymology 292, 456-473.
85. Hrycyna, C. A., Zhang, S., Ramachandra, M., Ni, B., Pastan, I., and Gottesman, M. M. (1996) Functional and Molecular Characterization of the Human Multidrug Transporter, in Multidrug Resistance in Cancer Cells (Gupta, S., and Tsuruo, T., Eds.), pp 29-37, John Wiley & Sons, Ltd., New York.
86. Ejendal, K. F., and Hrycyna, C. A. (2002) Multidrug resistance and cancer: the role of the human ABC transporter ABCG2, Current protein & peptide science 3, 503-511.
87. Bauer, B., Miller, D. S., and Fricker, G. (2003) Compound profiling for P-glycoprotein at the blood-brain barrier using a microplate screening system, Pharmaceutical research 20, 1170-1176.
88. Bauer, B., Yang, X., Hartz, A. M., Olson, E. R., Zhao, R., Kalvass, J. C., Pollack, G. M., and Miller, D. S. (2006) In vivo activation of human pregnane X receptor tightens the blood brain barrier to methadone through P-glycoprotein up-regulation, Molecular pharmacology 70, 1212-1219.
89. Fellner, S., Bauer, B., Miller, D. S., Schaffrik, M., Fankhanel, M., Spruss, T., Bernhardt, G., Graeff, C., Farber, L., Gschaidmeier, H., Buschauer, A., and Fricker, G. (2002) Transport of paclitaxel (Taxol) across the blood-brain barrier in vitro and in vivo, J Clin Invest 110, 1309-1318.
90. Audus, K. L., and Borchardt, R. T. (1987) Bovine brain microvessel endothelial cell monolayers as a model system for the blood-brain barrier, Annals of the New York Academy of Sciences 507, 9-18.
91. Franke, H., Galla, H., and Beuckmann, C. T. (2000) Primary cultures of brain microvessel endothelial cells: a valid and flexible model to study drug transport through the blood-brain barrier in vitro, Brain Research 5, 248-256.
92. Gutmann, H., Torok, M., Fricker, G., Huwyler, J., Beglinger, C., and Drewe, J. (1999) Modulation of multidrug resistance protein expression in porcine brain capillary endothelial cells in vitro, Drug metabolism and disposition: the biological fate of chemicals 27, 937-941.
93. Lechardeur, D., Phung-Ba, V., Wils, P., and Scherman, D. (1996) Detection of the multidrug resistance of P-glycoprotein in healthy tissues: the example of the blood-brain barrier, Annales de biologie clinique 54, 31-36.
94. Miller, D. S., Nobmann, S. N., Gutmann, H., Toeroek, M., Drewe, J., and Fricker, G. (2000) Xenobiotic transport across isolated brain microvessels studied by confocal microscopy, Molecular Pharmacology 58, 1357-1367.
95. Bauer, B., Hartz, A. M., Fricker, G., and Miller, D. S. (2004) Pregnane X receptor upregulation of P-glycoprotein expression and transport function at the blood-brain barrier, Molecular Pharmacology 66, 413-419.
96. Hartz, A. M., Bauer, B., Fricker, G., and Miller, D. S. (2004) Rapid regulation of Pglycoprotein at the blood-brain barrier by endothelin-1, Molecular Pharmacology 66, 387 394.
97. a) Pierson, T.; McArthur, J.; Siliciano, R. F. “Reservoirs for HIV-1: mechanisms for viral persistence in the presence of antiviral immune responses and antiretroviral therapy” Ann. Rev. 1 mm. (2000) 18, 665-708. b) Schrager, L. K.; D′Souza, M. P. “Cellular and anatomical reservoirs of HIV-1 in patients receiving potent antiretroviral combination therapy” J. Am. Med Assoc. (1998), 280, 67-71. c) Brack-Werner, R. “Astrocytes: HIV cellular reservoirs and important participants in neuropathogenesis” AIDS (1999) 13, 1-22.
98. Kaul, M.; Garden, G.; Lipton, S. “Pathways to neuronal injury and apoptosis in HIV-associated dementia” Nature (2001) 410, 988-994.
99. Glynn, S. L.; Yazdanian, M. “In vitro blood-brain barrier permeability of nevirapine compared to other HIV antiretroviral agents” J. Pharm. Sci. (1997) 87, 306-310.
100. Ramakrishnan, P. “The role of P-glycoprotein in the blood-brain barrier” Einstein Quart. J. Biol. Med. (2003) 19, 160-165.
101.a) Gottesman, M. M.; Pastan, I. “Biochemistry of multidrug resistance mediated by the multidrug transporter” Annu Rev Biochem (1993) 62, 385-427. B) Gottesman, M. M.; Hrycyna, C. A.; Schoenlein, P. V.; Germann, U. A.; Pastan, I. “Genetic analysis of the multidrug transporter” Annu Rev Genet (1995) 29, 607-649.
102.a) Hrycyna, C. A.; Ramachandra, M.; Germann, U. A.; Cheng, P. W.; Pastan, I.; Gottesman, M. M. “Both ATP sites of human P-glycoprotein are essential but not symmetric” Biochemistry (1999) 38, 13887-99. b) Loo, T. W.; Clarke, D. M. “Covalent modification of human P-glycoprotein mutants containing a single cysteine in either nucleotide-binding fold abolishes drug-stimulated ATPase activity” J Biol Chem (1995) 270, 22957-61.
103. Raviv, Y.; Pollard, H. B.; Bruggemann, E. P.; Pastan, I.; Gottesman, M. M. “Photosensitized labeling of a functional multidrug transporter in living drug-resistant tumor cells” J Biol Chem (1990) 265, 3975-80.
104. Bachmeier, C. J.; Spitzenberger, T. J.; Elmquist, W. F.; Miller, D. W. “Quanitative assessment of HIV-1 protease inhibitor interactions with drug efflux transporters of the blood-brain barrier” Pharm. Res. (2005) 22, 1259-1268.
105. Kim, R. B.; Fromm, M. F.; Wandel, C.; Leake, B.; Wood, A. J.; Roden, D. M.; Wilkinson, G. R. “The drug transporter P-glycoprotein limits oral absorption and brain entry of HIV-1 protease inhibitors” J. Clin. Invest. (1998) 101, 289-294.
106. Choo, E. F.; Leake, B.; Wandel, C.; Imamura, H.; Wood, A. J.; Wilkinson, G. R.; Kim, R. B. “Pharmacological inhibition of P-glycoprotein transport enhances the distribution of HIV-1 protease inhibitors into brain and testes” Drug Metab. Dispos. (2000) 28, 655-660.
107.a) Dey, S.; Ramachandra, M.; Pastan, I.; Gottesman, M. M.; Ambudkar, S. V. “Evidence for two nonidentical drug-interaction sites in the human P-glycoprotein” Proc Natl Acad Sci USA (1997) 94, 10594-9. b) Loo, T. W.; Bartlett, M. C.; Clarke, D. M. “Simultaneous binding of two different drugs in the binding pocket of the human multidrug resistance P-glycoprotein” J Biol Chem (2003) 278, 39706-10. c) Shapiro, A. B.; Ling, V. “Positively cooperative sites for drug transport by P-glycoprotein with distinct drug specificities” Eur J Biochem (1997) 250, 130-7.
108. Pires, M. M.; Hrycyna, C.; Chmielewski, J. “Bivalent inhibitors of the multidrug transporter P-glycoprotein” (2006) submitted.
109. Vierling, P.; Greiner, J. “Prodrugs of HIV protease inhibitors” Curr. Pharm. Des. (2003) 9, 1755-1770.
110. Wang. Q.; Rager, J. D.; Weinstein, K.; Kardos, P. S.; Dobson, G. L.; Li, J.; Hidalgo, I. J. “Evaluation of the MDR-MDCK cell line as a permeability screen for the blood-brain barrier” Int. J. Pharm. (2005) 288, 349-359.
111. Williams, G.; Knipp, G.; Sinko, P. “The effect of cell culture conditions on saquinavir transport through, and interactions with, MDCKII cells overexpressing hMDR1” J. Pharm. Sci. (2003) 92, 1957-1967.

We claim:

1. A method for inhibiting therapeutic drug resistance within a cell over-expressing a membrane protein, comprising:
synthesizing a dimeric prodrug inhibitor of a monomeric therapeutic agent;
administering the dimeric prodrug inhibitor to the membrane protein together with the monomeric therapeutic agent; and
occupying at least one substrate binding site of the membrane protein with the synthesized dimeric prodrug to allow the monomeric therapeutic agent to accumulate within the cell;
wherein the dimeric prodrug inhibitor contains a crosslinking agent that is adapted to breakdown under reducing conditions within the cytosol of the cell to cause the dimeric prodrug to revert back to a form equivalent to the monomeric therapeutic agent.

2. The method of claim 1, wherein administering the dimeric prodrug inhibitor to a membrane protein comprises administering the dimeric prodrug inhibitor to a multidrug resistance transporter protein selected from the group consisting of P-glycoprotein and ABCG2.

3. The method of claim 1, wherein the crosslinking agent comprises a traceless linker.

4. The method of claim 3, wherein the traceless linker is a tether containing an internal disulfide with ester, carbonate or carbamate linkages to the monomeric therapeutic agent.

5. The method of claim 4, wherein the disulfide is adapted to reduce to a thiol moiety under the reducing conditions, the thiol moiety being adapted to act as a nucleophile to cleave the linkages to the monomeric therapeutic agent and release the tether as a non-toxic by-product.

6. The method of claim 1, wherein the monomeric therapeutic agent is selected from the group consisting of abacavir, zidovudine, amprenavir, morphine, topotiramate, perphenazine, quetiapine, quinidine, quinine, saquinavir, indinavir, nelfinavir, venlafaxine, ritonavir, phenytoin, carbamazepine, phenobarbital, lamotrigine, oxcarbazepine, galantamine and imatinib mesylate.

7. The method of claim 1, wherein the step of reverting the prodrug back to a monomeric form that is equivalent to the therapeutic agent comprises subjecting the prodrug to esterase.

8. The method of claim 1, wherein the step of breaking down the crosslinking agent under reducing conditions comprises subjecting the crosslinking agent to at least one of dithiothreitol and glutathione.

9. The method of claim 1, wherein the cell includes at least one of a brain cell, a testes cell, a macrophage and a lymphocyte.

10. A compound of formula (I) 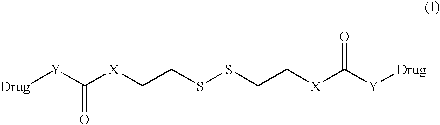
wherein Drug is a monomeric therapeutic agent selected from the group consisting of abacavir, zidovudine, amprenavir, morphine, phenytoin, and venlafaxine; X is CH₂ or O; and Y is O, N or aldehyde/ketone adduct.
